(19) United States
(12) Patent Application Publication
(10) Pub. No.: US 2017/0340852 A1
(43) Pub. Date: Nov. 30, 2017

(54) Infection Containment Band (IC Band)

(71) Applicant: Mark Deitrickson, Kansas City, MO (US)

(72) Inventor: Mark Deitrickson, Kansas City, MO (US)

(21) Appl. No.: 15/603,890

(22) Filed: May 24, 2017

Related U.S. Application Data
(60) Provisional application No. 62/340,965, filed on May 24, 2016.

(51) Int. Cl. A61M 16/10; B01D 46/10; B01D 46/00

(52) U.S. Cl. CPC A61M 16/1055; B01D 46/0028; B01D 46/10; A61M 2205/7518; A61M 2205/7509; A61M 2205/125; A61M 2210/083; A61M 2209/088

(57) ABSTRACT

An infection containment band (IC Band) that consists of a wrist band with a mouth sized cavity that holds a filter in the filter chamber which is attached to, and/or part of the wrist band-resembling a wrist watch. There are pressure vents on the unit that release the air pressure when coughing or sneezing into the unit. The filter, in preferred embodiments consists of a sub-micron and/or standard filter and/or anti-microbial filter. The foregoing and other objects are intended to be illustrative of the invention and are not meant in a limiting sense. Many possible embodiments of the invention may be made and will be readily evident upon a study of the following specification and accompanying drawings comprising a part thereof. Various features and sub-combinations of invention may be employed without reference to other features and sub-combinations. Other objects and advantages of this invention will become apparent.

### FIELD OF THE INVENTION

The present invention relates to devices and methods for reducing the spread of viral, bacterial and/or mycobacterial micro-organisms as a result of coughing or sneezing. It gives adults and children a real solution to contain and reduce the spread of infectious germs when coughing or sneezing (especially containing submicron germs; which cannot be contained through traditional safety measures), e.g., using their hand, arm, paper product, cloth product or mask.

### BACKGROUND OF THE INVENTION

According to the CDC and health care experts like former US Surgeon General David Satcher M.D., coughing and sneezing are the primary ways to spread colds, the flu and most contagious diseases. Dr. Satcher states; Colds and Flu's are mainly spread by inhaling aerosolized droplets that come into contact with our mucus membranes such as when someone coughs or sneezes. We breathe in the viral or bacterial germs.

When people cough or sneeze, they may sneeze or cough into the air or they may use their hand, elbow, Kleenex (paper product) or cloth product.

The problem is that any of these current remedies will result in the contamination of the air. Even after using a paper product or cloth product to contain the sneeze or cough.

Two of the biggest hurdles to overcome in reducing the spread of infectious materials due to coughing and /or sneezing are; 1) having a solution right there when a spontaneous cough or sneeze occurs, and containing the germs. 2) Most of these germs are sub-micron in size; meaning they will pass right through clothing, handkerchiefs, tissue, masks and any method to try and contain the cough or sneeze. These sub-micron particles are like a gas, can't be seen by the eye and can stay airborne for hours. They are the most dangerous elements of a cough and/or sneeze.

According to Lydia Bourouiba, head of the Fluid Dynamics of Disease Transmission Laboratory at MIT; coughs and sneezes produce clouds of invisible gas that carry infectious droplets up to 200 times farther than they would have traveled if they were simply disconnected drops.

Eventually the airborne particles settle and contaminate surfaces. That's when the germs are spread due to touching surfaces.

Viruses and bacteria can live on surfaces for hours so re-contamination is easy. According to the CDC website “Spread of 2009 H1N1 virus occurs in the same way that seasonal flu spreads. Flu viruses are spread mainly from person to person through coughing or sneezing by people with influenza”. These germs move from one person to another or to many others and then exponentially a viral or bacterial germ moves through the population.

The negative effects of this have been lost productivity for workers and companies, lost time for education for teachers and students, hospitalization and even death for some of the population.

According to the CDC, respiratory infections cost billions of dollars to the US economy through lost worker productivity and health care costs.

World Bank estimates that a severe influenza (like H1N1) could kill 71 million people globally and cause a recession costing more than $3 trillion.

Tuberculosis is spread the same way as a cold or flu and it's the most prevalent respiratory infection in the world.

50 million Americans catch the flu each year. 35,000 Americans dies each year from the seasonal flu. Health care cost in the US from infectious disease is estimated to be 120 Billion a year. The economic burden to US employers is estimated at 225 Billion per year.

The CDC hasn't changed any options for reducing the spread of viruses for 50 years except for coughing in the fold of your arm. There are two steps required to reduce the spread of viral, bacterial and mycobacterium germs through coughing and sneezing; 1) Getting to your mouth (immediately) due to the spontaneous nature of a cough. 2) Containing all the germs (large and small, especially the submicron germs/gas cloud).

Through years of research, testing and prototyping, the Infection Containment Band (IC Band) was developed to upgrade the current method for reducing the spread of viral and bacterial infections based on the CDC's best recommendations which is; cough in the fold of your arm. It's the first real “implementable” upgrade in disease spread prevention since the CDC first introduced their “Cover Your Cough” campaign. It's a band that is worn on the wrist so it's there when the spontaneity of a cough happens and it can accommodate a special submicron filter in a vented chamber to contain the submicron germs (gas cloud).

A square pouch (paper product) that you squeeze to open and you keep in your pocket or purse; a hand held mask with a filter that resides in a holster; a tissue and a handkerchief.

The other solutions do not contain the real threat in spreading infectious disease through coughing and sneezing; the invisible gas cloud of infectious material (submicron particles).

Submicron particles from coughing and sneezing are the most dangerous according to new studies. Submicron particles pass through clothing, cloth, tissue and are not contained by conventional methods of covering your cough. Submicron particles are comparable to a invisible gas. The other solutions are not capable of dealing with submicron particles.

Most coughing or sneezing is spontaneous and the person doesn't have time to grab a tissue, pull a mask with a filter out of a holster or grab an item from your pocket, squeeze it and then cough or sneeze into it. A solution needs to be there where you need it and it needs to contain the infectious material. These other solutions are not there when you need them (which is why most people try to cover their cough or sneeze with their elbow or hand) and they don't contain the infectious material. In any city in the US you'll notice that Americans won't carry and wear a mask or mask in a holster device because it carries a stigma.

The biggest shortcomings is that none of these options contain the submicron germs from a cough or sneeze which pass through regular filters, clothes, paper, etc., and they are not available for the spontaneity of coughing and sneezing.

### SUMMARY OF THE INVENTION

In accordance with the present invention, there is provided an infection containment band (IC Band) that consists of a mouth sized cavity (filter chamber) that holds a filter and/or submicron filter and/or anti-microbial filter. The filter chamber is attached to, and/or part of the wrist band—resembling a wrist watch. There are vents on the unit and/or the filter chamber that release the air pressure when coughing or sneezing into the unit. The filter resides in the filter chamber. The filter, in preferred embodiments consists of a sub-micron and/or standard filter and/or anti-microbial filter. The foregoing and other objects are intended to be illustrative of the invention and are not meant in a limiting sense. Many possible embodiments of the invention may be made and will be readily evident upon a study of the following specification and accompanying drawings comprising a part thereof. Various features and sub-combinations of invention may be employed without reference to other features and sub-combinations. Other objects and advantages of this invention will become apparent from the following description taken in connection with the accompanying drawings, wherein is set forth by way of illustration and example, an embodiment of this invention and various features thereof.

It would be advantageous to provide apparatuses and/or methods that reduce the spread of germs when coughing or sneezing occurs.

It would also be advantageous to provide apparatuses that people will want to use because the apparatus is right there when a person needs it for spontaneous coughing or sneezing while discretely blending into their lifestyle.

It would also be advantageous to provide apparatuses that people will want to use because the apparatus has the potential to create new options to protect ourselves from germs as a population.

It would further be advantageous to provide apparatuses that will contain the invisible submicron gas cloud of germs that can't be contained by traditional options.

It would also be advantageous to provide apparatuses that people will want to use because it's functionally a better solution than any alternative when coughing or sneezing.

It would also be advantageous to provide apparatuses that people will want to use because it's functionally a better solution than the CDC's current best solution of coughing or sneezing into your elbow.

It would further be advantageous to provide apparatuses that can reduce the occurrences of respiratory viruses due to its ability to reduce the spread of the viral and bacterial germs.

It would further be advantageous to provide apparatuses that can reduce the deaths due to respiratory viruses by reducing the spread of the viral and bacterial germs.

### BRIEF DESCRIPTION OF THE DRAWINGS

A complete understanding of the present invention may be obtained by reference to the accompanying drawings, when considered in conjunction with the subsequent, detailed description, in which:

FIG. 1 is a top perspective view of an embodiment of a completely disposable infection containment band (ic band) of the instant invention with a filter chamber that can hold a filter secured in the filter chamber showing various embodiments of vents on the side of the filter chamber, a band loop to secure the end of the band after closure and a chamber cap that secures the filter in the chamber. the (ic band) according to the invention is for the reduction of the spread of germs due to coughing and sneezing. the (ic band) according to the invention may be made from any materials commonly used in the art;

FIG. 2 is a top perspective view of an infection containment band (ic band) of FIG. 1, showing various embodiments of the different sizes and closure mechanisms;

FIG. 3 is a top perspective view of an infection containment band (ic band) of FIG. 1, showing various embodiments of the filter chamber venting options;

FIG. 4 is a top exploded view of an another embodiment of a infection containment band (ic band) of the instant invention utilizing a filter mesh over the various embodiments of the filter chamber venting;

FIG. 5 is a top exploded view of a rendered view of infection containment band (ic band) of FIG. 1, showing various embodiments of the vented filter chamber, the closure pin, the chamber cap and filter;

FIG. 6 is a bottom perspective view of an infection containment band (Ic band) of FIG. 1, showing various embodiments of the band and filter chamber as one piece from the back;

FIG. 7 is a top perspective view of an another embodiment of a infection containment band (ic band) of the instant invention utilizing different mechanics so the bands and the filter chambers can be mixed and matched; another embodiment of a infection containment band (ic band) of the instant invention utilizing different mechanics so the bands and the filter chambers can be mixed and matched;

FIG. 8 is a bottom perspective view of an infection containment band (ic band) of FIG. 7, showing various embodiments of the band and filter chamber;

FIG. 9 is a top perspective view of an infection containment band (ic band) of FIG. 7, showing various embodiments of the band closure options;

FIG. 10 is a right perspective view of an another embodiment of a infection containment band (ic band) of the instant invention utilizing a “slip on” band;

FIG. 11 is a top perspective view of an another embodiment of an infection containment band (ic band) of the instant invention utilizing different mechanics with a disposable filter cartridge secured in the filter cartridge chamber which utilizes a filter cartridge release button to discard the filter and showing various embodiments of the vents on the side of the ic band;

FIG. 12 is a top exploded view of an infection containment band (ic band) of FIG. 11, showing various embodiments of the filter cartridge attached to the top and how it snaps into the filter cartridge chamber and how the filter cartridge is released from the filter cartridge chamber; and

FIG. 13 is a top perspective view of an another embodiment of a infection containment band (ic band) of the instant invention utilizing different materials to the embodiments shown in FIGS. 11 & 12.

For purposes of clarity and brevity, like elements and components will bear the same designations and numbering throughout the Figures.

### DESCRIPTION OF THE PREFERRED EMBODIMENT

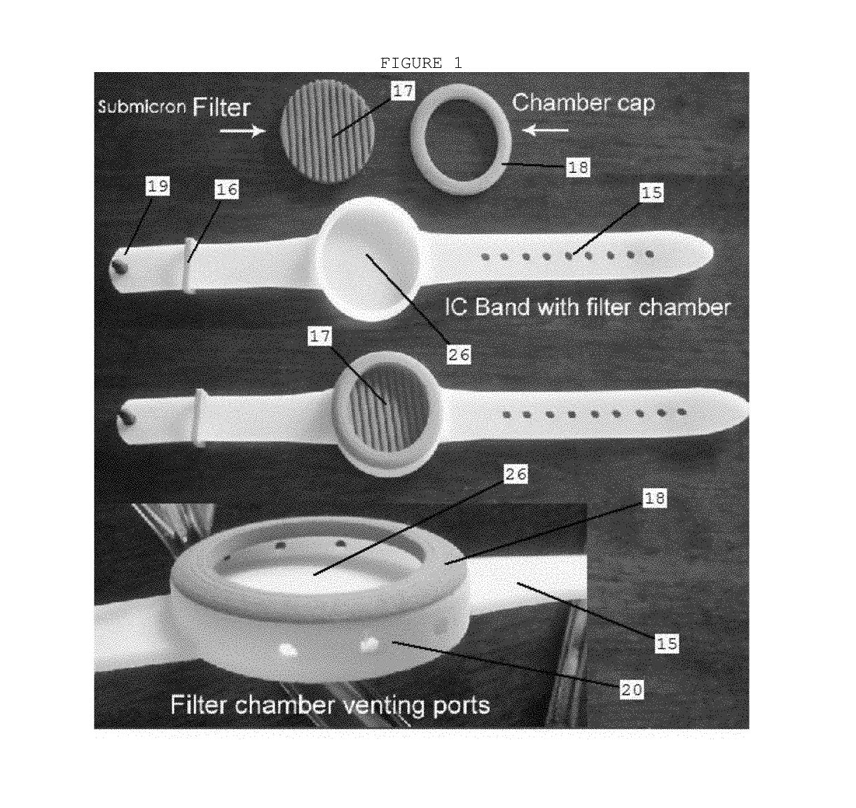

FIG. 1 is a top perspective view of an embodiment of a completely disposable infection containment band (IC Band) (#15) of the instant invention. The infection containment band is comprised of a wrist band with a vented filter chamber 26, a band loop 16 (#16) to secure the end of the band after closure and a chamber cap (#18) that secures the filter 17 (#17) in the chamber. The filter cartridge chamber 37 is shallow, allowing a submicron filter 17 (#17), anti-microbial filter 17 (#17) and/or submicron anti-microbial filter 17 (#17) to reside in the filter cartridge chamber 37 securely. In the embodiment shown, air vents on the sides of the filter cartridge chamber 37 allow for pressure to be released when coughing or sneezing into the infection containment band.

It will be appreciated that the IC Band (#15) can be similar to an adjustable watch band, made of cloth, rubber, plastic, leather or other non-metal material, a metal link band which may or may not have a clasp to open it or any mechanism to open or close it, or a slip on band or any type of band or strap made from any material may be utilized without departing from the spirit and scope of the instant invention.

The IC Band (#15) according to the invention is for the reduction of the spread of germs due to coughing and sneezing. The IC Band (#15) according to the invention may be made from any materials commonly used in the art.

The filter chamber 26 can accommodate any type of filter 17 (#17) material. The filter 17 (#17) media is used to trap/contain infectious material as a result of coughing and/or sneezing into the IC Band (#15).

The filter chamber cap 18 (#18) is attached to the filter chamber 26 (#15) by gluing, welding, or mechanically fastening in a manner well known to those skilled in the art. It will be appreciated that the filter chamber cap 18 (#18) and the filter chamber 26 (#15) can be one piece instead of two separate pieces as referenced above.

Although the face of the filter chamber cap 18 (#18) and filter chamber 26 (#15) are shown as generally oval, it will be appreciated that the filter chamber cap 18 (#18) and filter chamber 26 (#15) may be oval, round, square or any shape desired. Nevertheless, it will also be appreciated that other materials may be utilized without departing from the spirit and scope of the instant invention.

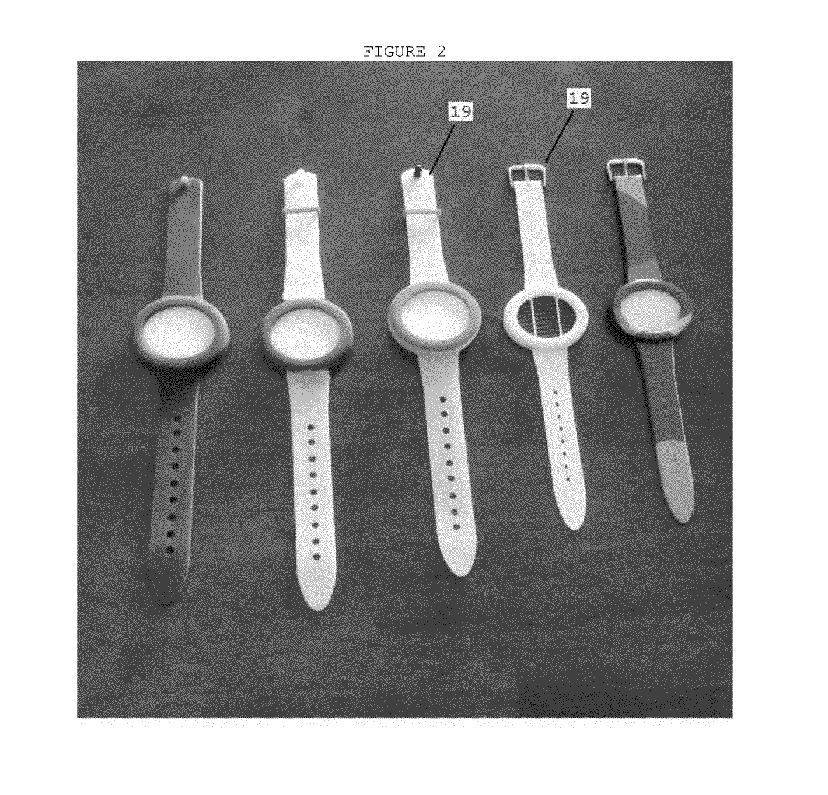

FIG. 2 is a top perspective view of the infection containment band (IC Band) (#15) of FIG. 1, showing various embodiments of the different sizes and closure mechanisms. It will be appreciated that the IC Band (#15) can utilize any type of closure mechanism (#19) for fastening an IC Band (#15), made from any material well known to those skilled in the art without departing from the spirit and scope of the instant invention.

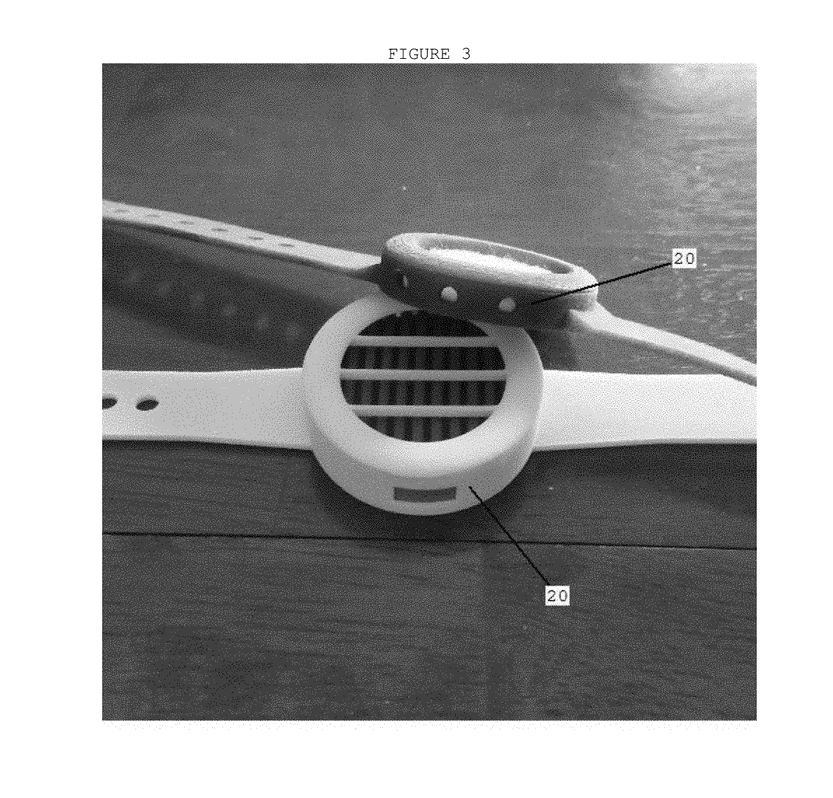

FIG. 3 is a top perspective of the IC Band (#15) of FIG. 1, showing various embodiments of the filter chamber 26 venting (#20) options. It will be appreciated that the IC Band (#15) can utilize any shape or location of the venting without departing from the spirit and scope of the instant invention.

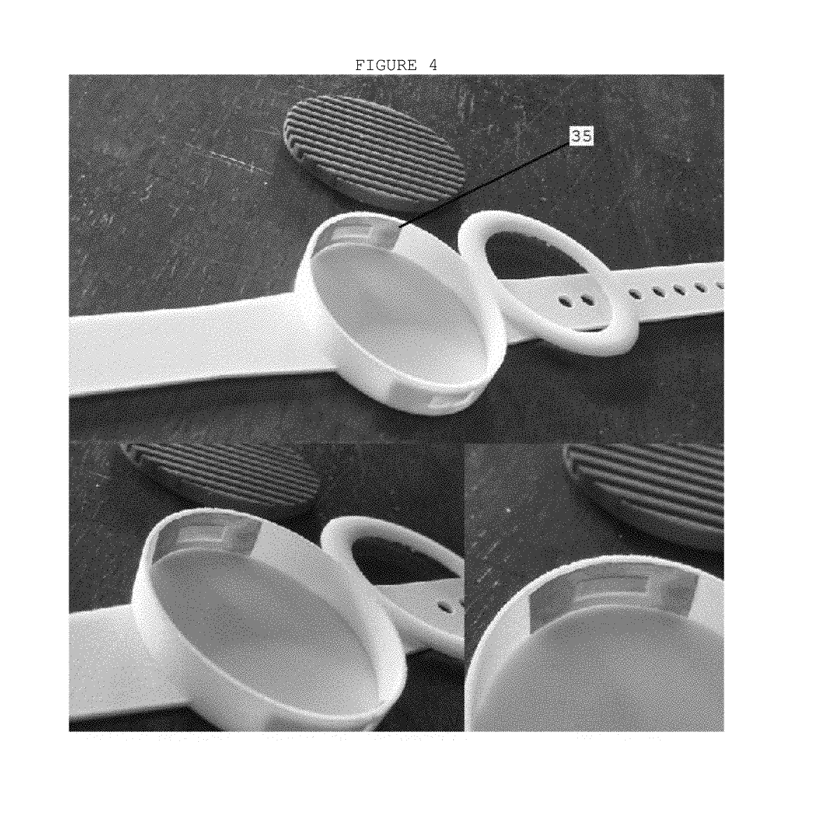

FIG. 4 is a top exploded view of another embodiment of the IC Band (#15) of the instant invention showing different mechanics utilizing a filter 17 mesh (#35) to cover the venting ports (#20). The filter 17 mesh (#35) is attached to the vent 29 ports (#20) on the filter chamber 26 (#15) by gluing, welding, or mechanically fastening in a manner well known to those skilled in the art. The filter 17 mesh (#35) can be of any micron size, made from any suitable material and is designed to contain the particles of infectious material from coughing and sneezing.

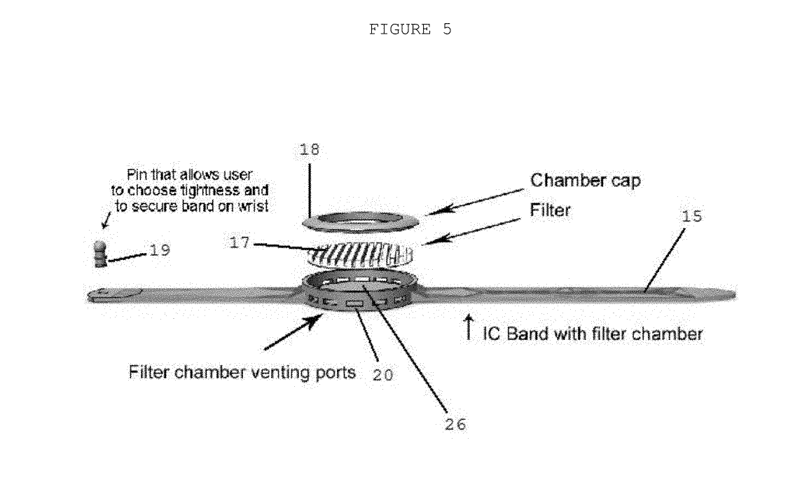

FIG. 5 is a top exploded engineered view of the IC Band (#15) FIG. 1, showing various embodiments of the band with the vented filter chamber 26 (#15), the chamber cap (#18), the filter 17 (#17) and the closure pin (#19).

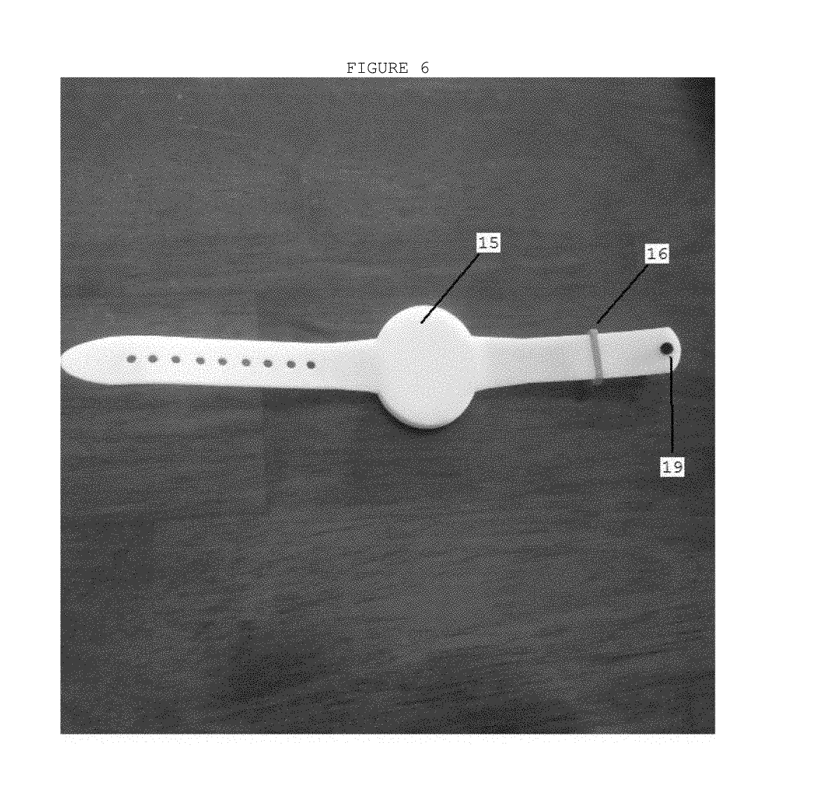

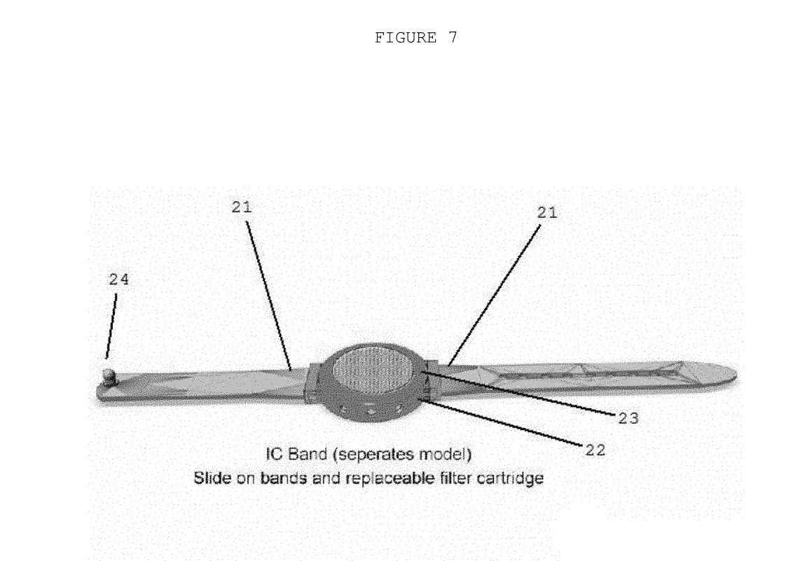

FIG. 6 is a bottom perspective of the IC Band (#15) of FIG. 1, showing various embodiments of the band and filter chamber 26 (#15) as one piece, including the chamber cap (#18), the band loop 16 (#16) and closure pin (#19). The band and the filter chamber 26 (#15) can be one piece or separate pieces (see FIG. 7) and attached in any manner or fashion without departing from the spirit and scope of the instant invention.

FIG. 7 is a top perspective of another embodiment of an IC Band of the instant invention utilizing different mechanics so the bands (#21) and the filter chamber 26 (#22) and filter chamber cap 18 (#23) can be mixed and matched. It will be appreciated that the band can utilize any type of fastening mechanism for fastening the wrist band (#21) to the filter chamber 26 (#22), made from any material well known to those skilled in the art without departing from the spirit and scope of the instant invention.

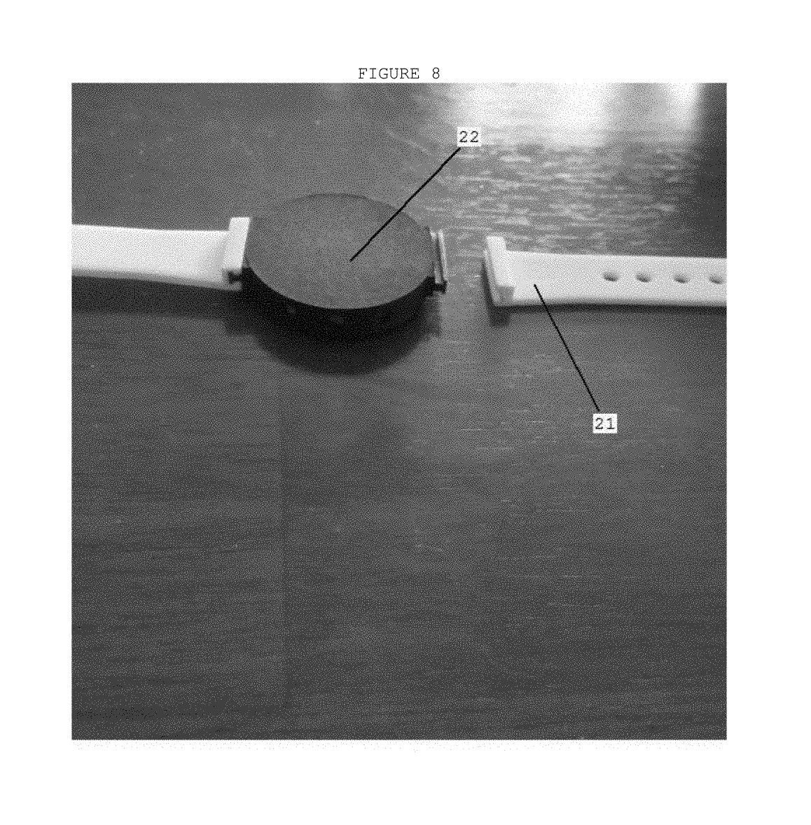

FIG. 8 is a bottom perspective of an IC Band of FIG. 7, showing various embodiments of the band (#21) and filter chamber 26 (#22).

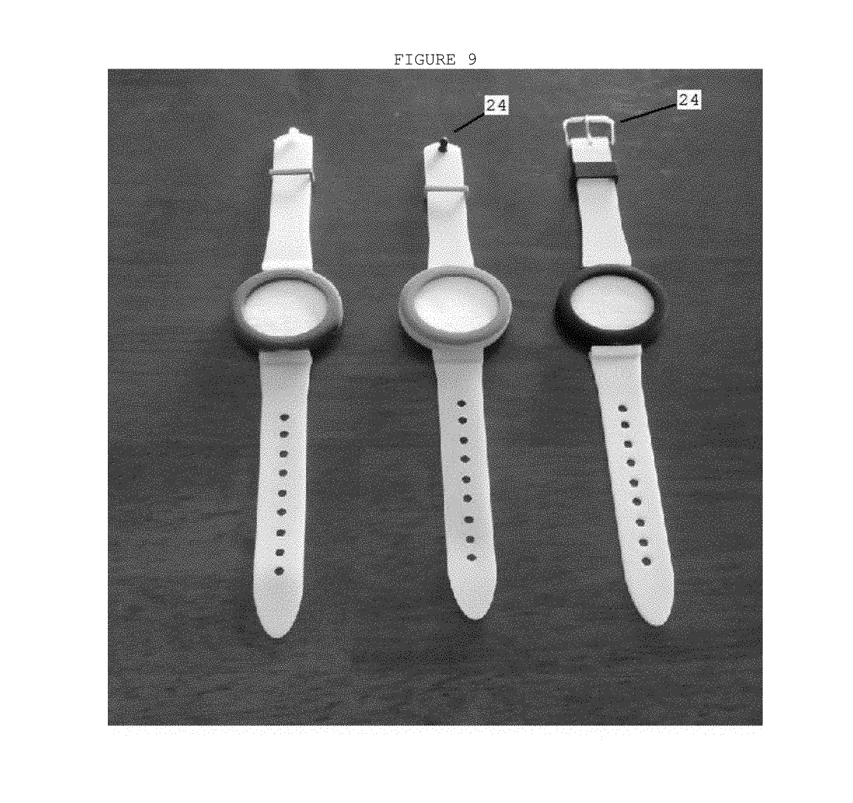

FIG. 9 is a top perspective of an IC Band of FIG. 7, showing various embodiments of the band closures options (#24). It will be appreciated that the IC Band of FIG. 7, can utilize any type of closure mechanism (#24) for fastening a wrist band (#21), made from any material well known to those skilled in the art without departing from the spirit and scope of the instant invention.

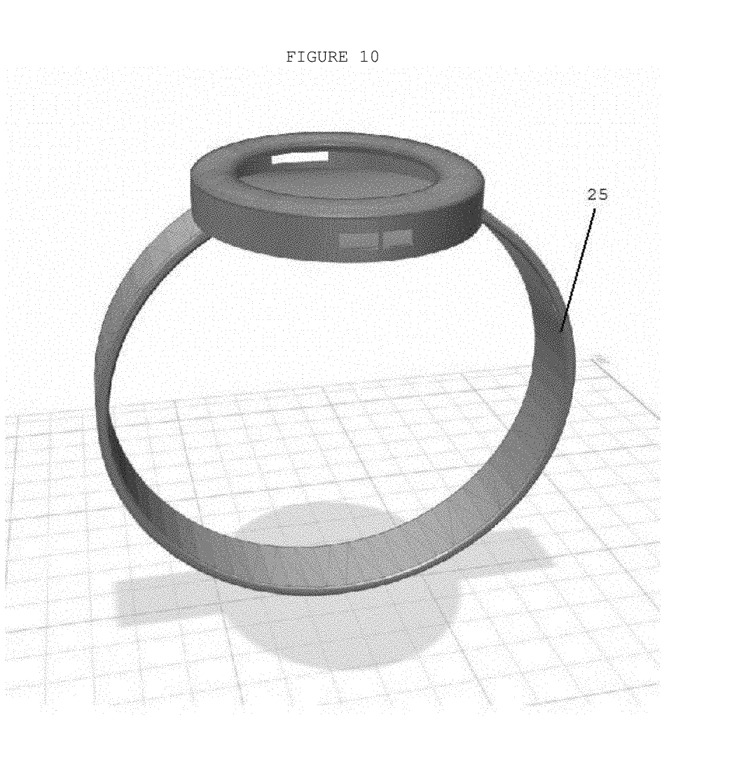

FIG. 10 is a right perspective of another embodiment of an IC Band of the instant invention utilizing a “slip on” band. It will be appreciated that the entire slip-on IC Band 25 (#25) is one piece or can be a multi-piece unit without departing from the spirit and scope of the instant invention.

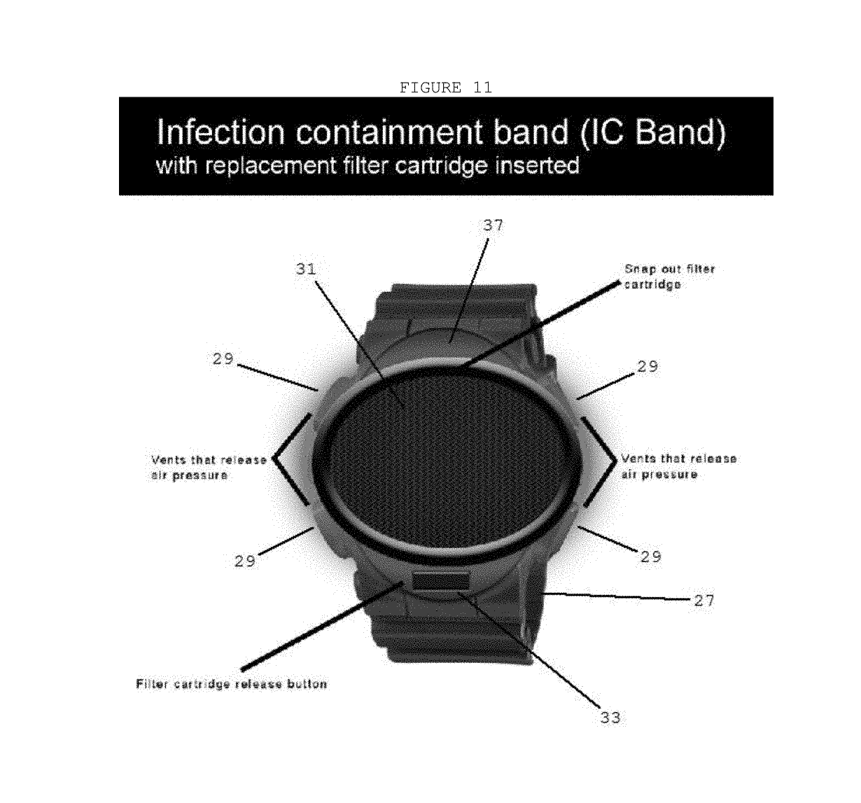

FIG. 11 is a top perspective view of an another embodiment of an IC Band (#27) of the instant invention utilizing different mechanics with a disposable filter cartridge 31 (#31) secured in the filter cartridge chamber 37 (#37) which utilizes a filter cartridge release button 33 (#33) to discard the filter 17 (#31). In the embodiment shown, air vents (#29) on the sides of the filter cartridge chamber 37 allow for pressure to be released when coughing or sneezing into the submicron and/or anti-microbial and/or submicron anti-microbial filter 17 (#31).

It will be appreciated that the IC Band (#27) can be similar to an adjustable watch band, made of cloth, rubber, plastic, leather or other non-metal material, a metal link band which may or may not have a clasp to open it or any mechanism to open or close it, or a slip on band or any type of band or strap made from any material without departing from the spirit and scope of the instant invention.

The IC Band (#27) according to the invention is for the reduction of the spread of germs due to coughing and sneezing.

The filter cartridge chamber 37 (#37) can accommodate any type of filter cartridge 31 (#31) material. This filter cartridge 31 (#31) media is used to trap/contain infectious material as a result of coughing and/or sneezing into the IC Band (#27).

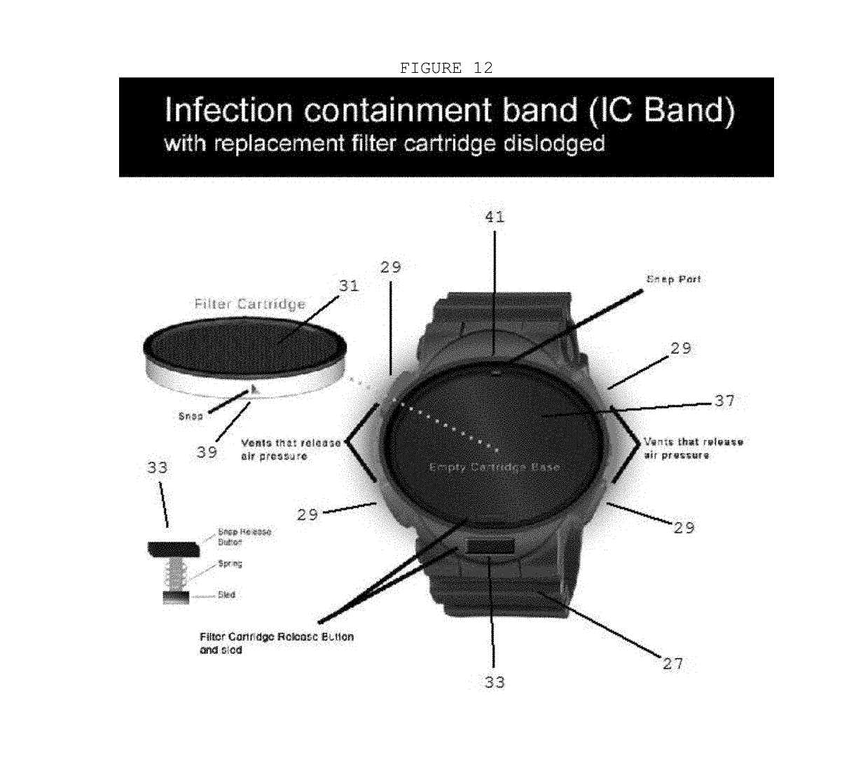

FIG. 12 is an exploded view of the IC Band (#27) of FIG. 11, showing various embodiments of the filter cartridge 31 (#31) and the filter cartridge chamber 37 (#37). It will be appreciated that the filter cartridge chamber 37 (#37) has a filter cartridge 31 (snap 39) release button (#33) on the side of the filter cartridge chamber 37 (#37). In the embodiment shown, the snap 39 release button (#33) utilizes a spring that pushes a plastic sled up and down which pops out the filter cartridge 31 (#31) when secured in the chamber. It will also be appreciated that the (snap 39) release button (#33) can be made from any material and utilize any reasonable method to release the filter cartridge 31 (#31)from the filter cartridge chamber 37 (#37).

It will also be appreciated that the filter cartridge 31 (#31) has a protruding snap 39 (#39) on the side of the filter cartridge 31 (#31) that allows the filter cartridge 31 (#31) to snap 39 (#39) securely into the snap port 41 (#41) inside the filter cartridge chamber 37 (#37) or any reasonable method to secure the filter cartridge 31 (#31) into the filter cartridge chamber 37 (#37). The snap port 41 (#41), located on the inside wall of the filter cartridge chamber 37 (#37) is a small cavity that allows the snap 39 (#39), on the filter cartridge 31 (#31) to plug into the snap port 41 (#41) and secure the filter cartridge 31 (#31) inside the unit. It will also be appreciated that the filter cartridge 31 (#31) has a rim attached to the top of the filter cartridge 31 (#31) in an effort to create a better seal when coughing or sneezing into the unit and can be made from rubber or any reasonable material without departing from the spirit and scope of the instant invention.

In a preferred embodiment, the filter 17 (#17) pad is constructed from a submicron filter 17 (#17) media that hospitals currently use to filter 17 viruses and/or bacterial germs. New data from MIT shows the most dangerous germs are submicron and it is recommended that the use of the submicron filter 17 media (#17) would be preferred. Nevertheless, it will also be appreciated that other materials maybe utilized without departing from the spirit and scope of the instant invention. For example, in one embodiment, the pad is simply an absorbent material to help reduce the spread of fluids from the sneeze or cough similar to the use of a tissue or handkerchief. In another embodiment, the pad is a material that is treated with an anti-viral and/or anti-bacterial agent. In another embodiment, the pad is a submicron material that is rated to stop the smallest viral or bacterial materials that are submicron/in a gas form.

The IC Band (#27) according to the invention is for the reduction of the spread of germs due to coughing and sneezing. The IC Band (#27) according to the invention may be made from any materials commonly used in the art.

Although the face of the filter cartridge 31 (#31) and filter cartridge chamber 37 (#37) are shown as generally oval, it will be appreciated that the filter cartridge 31 (#31) and filter cartridge chamber 37 (#37) may be oval, round, square or any shape desired.

It will be appreciated that the IC Band (#27) can be similar to an adjustable watch band, made of cloth, rubber, leather or other non-metal material, a metal link band which may or may not have a clasp to open it or any mechanism to open or close it, or a slip on band or any type of band or strap made from any material.

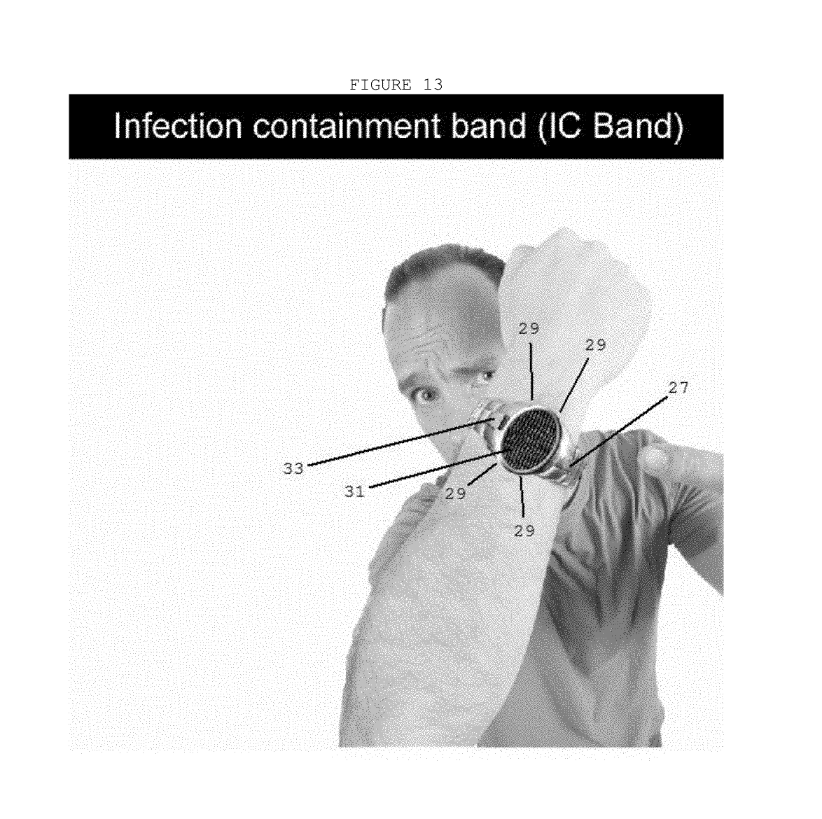

FIG. 13 is a top view of another embodiment of an IC Band (#27) that utilizes different materials to the embodiments shown in FIGS. 11 & 12. It will be appreciated that the invention may be made from any materials commonly used in the art, allowing the IC Band (#27) to fit into anyone's lifestyle.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A completely -disposable infection containment band (ic band) worn on the wrist for capturing and containing the infectious aerosolized droplets (viral, bacterial and/or mycobacterial micro-organisms) from a sick persons cough and/or sneeze, comprising:
(a) one piece wrist band with filter chamber which holds a sub-micron filter, anti-microbial filter or standard filter;
(b) filter chamber, rigidly connected to ic band;
(c) filter chamber cap for securing the filter in place and creating a smooth rim for the mouth, separately connected to ic band;
(d) vents around filter chamber for releasing pressure from filter chamber when someone coughs or sneezes into the ic band thus reducing infectious material blow back, rigidly connected to ic band;
(e) band loop for keeping the ic band secure around the wrist, separately connected to ic band;
(f) locking mechanism for closing and locking the ic band in place around the wrist, rigidly connected to ic band;
(g) sub-micron filter to capture sub-micron gas cloud from cough or sneeze, separately connected to ic band;

2. Another embodiment of the infection containment band (ic band) venting, comprising:
(a) sub-micron filter mesh securely fastened over the various embodiments of the filter chamber venting;

3. Another embodiment of the infection containment band (ic band) with an interchangeable wrist band and filter chamber worn on the wrist for capturing and containing the infectious aerosolized droplets (viral, bacterial and/or mycobacterial micro-organisms) from a sick persons cough and/or sneeze, comprising:
(a) interchangeable wrist band, lockably interconnected;
(b) interchangeable filter chamber which holds a sub-micron filter, anti-microbial filter or standard filter, lockably interconnected;
(c) filter chamber cap for securing the filter in place and creating a smooth rim for the mouth, separately connected to ic band;
(d) vents around filter chamber for releasing pressure from filter chamber when someone coughs or sneezes into the ic band thus reducing infectious material blow back, rigidly connected to ic band;
(e) band loop for keeping the ic band secure around the wrist, separately connected to ic band;
(f) locking mechanism for closing and locking the ic band in place around the wrist, rigidly connected to ic band;
(g) sub-micron filter to capture sub-micron gas cloud from cough or sneeze, separately connected to ic band;

4. Another embodiment of a completely disposable slip-on infection containment band (ic band) worn on the wrist for capturing and containing the infectious aerosolized droplets (viral, bacterial and/or mycobacterial micro-organisms) from a sick persons cough and/or sneeze, comprising:
(a) one piece wrist band with filter chamber which holds a sub-micron filter, anti-microbial filter or standard filter;
(b) filter chamber, rigidly connected to ic band;
(c) filter chamber cap for securing the filter in place and creating a smooth rim for the mouth, separately connected to ic band;
(d) vents around filter chamber for releasing pressure from filter chamber when someone coughs or sneezes into the ic band thus reducing infectious material blow back, rigidly connected to ic band;
(e) band loop for keeping the ic band secure around the wrist, separately connected to ic band;
(f) locking mechanism for closing and locking the ic band in place around the wrist, rigidly connected to ic band;
(g) sub-micron filter to capture sub-micron gas cloud from cough or sneeze, separately connected to ic band;

5. Another embodiment of the infection containment band (ic band) with a disposable filter cartridge worn on the wrist for capturing and containing the infectious aerosolized droplets (viral, bacterial and/or mycobacterial micro-organisms) from a sick persons cough and/or sneeze, comprising:
(a) one piece wrist band with filter chamber which holds a sub-micron filter, anti-microbial filter or standard filter cartridge;
(b) filter chamber, rigidly connected to ic band;
(c) vents around filter chamber for releasing pressure from filter chamber when someone coughs or sneezes into the ic band thus reducing infectious material blow back, rigidly connected to icband;
(d) band loop for keeping the ic band secure around the wrist, separately connected to ic band;
(e) locking mechanism for closing and locking the ic band in place around the wrist, rigidly connected to ic band;
(g) disposable filter cartridge, separately connected to ic band;
(h) filter cartridge release button to discard used filter cartridge, securely attached to filter chamber;
